(19) United States
(12) Patent Application Publication
(10) Pub. No.: US 2014/0350285 A1
(43) Pub. Date: Nov. 27, 2014

(54) NOVEL POLYMORPHS OF N-[2-AMINO-4-(4-FLUOROBENZYLAMINO)-PHENYL] CARBAMIC ACID ETHYL ESTER AND PROCESSES THEREOF

(71) Applicants: Dodda Mohan Rao, Hyderabad (IN); Kirla Haritha, Pamarru (IN)

(72) Inventors: Dodda Mohan Rao, Hyderabad (IN); Kirla Haritha, Pamarru (IN)

(73) Assignee: SYMED LABS LIMITED, Hyderabad (IN)

(21) Appl. No.: 14/372,506

(22) PCT Filed: Jan. 30, 2012
(86) PCT No.: PCT/IN12/00063

§ 371 Date: Jul. 16, 2014

(51) Int. Cl. C07C 269/08; C07C 271/28

(52) U.S. Cl. CPC C07C 269/08; C07C 271/28; C07B 2200/13
USPC 560/27

(57) ABSTRACT

The present invention relates to novel polymorphs of N-[2-amino-4-(4-fluorobenzylamino)-phenyl]carbamic acid ethyl ester, processes for preparing them, and pharmaceutical composition comprising them. In one aspect, the present invention relates to a novel crystalline polymorph of retigabine designated as crystalline Form I, characterized by XRPD having characteristic peaks at about 4.87, 5.04, 7.03, 9.74, 10.02, 11.6, 18.03, 19.9 and 28.5±0.2 degrees two-theta, which is substantially same as depicted in FIG. 1.

### FIELD OF THE INVENTION

The present invention relates to novel polymorphs of N-[2-amino-4-(4-fluorobenzylamino)-phenyl]carbamic acid ethyl ester and processes thereof.

### BACKGROUND OF THE INVENTION

Retigabine is a pharmaceutical substance with anticonvulsive, antipyretic and analgesic activity. Retigabine is being approved in US and Europe for the treatment of Epilepsy. It is marketed under the Brand names Potiga in US and Trobalt in Europe in the form of immediate release film coated tablets in dosage strengths of 50, 100, 200, 300 and 400 mg. Retigabine is also called as Ezogabine and chemically described as N-[2-amino-4-(4-fluorobenzylamino)-phenyl]carbamic acid ethyl ester (herein after referred by generic name retigabine) and is represented by the structural formula I:

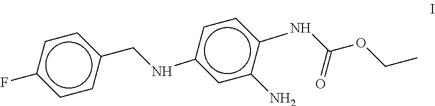

U.S. Pat. No. 5,384,330 discloses retigabine, a pharmaceutically acceptable acid addition salt thereof, a pharmaceutical composition and a method of treatment thereof.

The US '330 patent also describes a process for the preparation of retigabine as exemplified in example 1. In this example, retigabine is obtained as its dihydrochloride salt upon recrystallization from ethanol/ether. No information about polymorph form of retigabine is given.

U.S. Pat. No. 6,538,151 discloses three different crystalline forms of retigabine, denominated therein as modifications A, B, and, C, which are characterized by XRPD 2-theta, FTIR and DSC. The US '151 patent also describes processes for preparing the crystalline forms of retigabine and pharmaceutical compositions comprising the crystalline forms.

International Patent application publication No. WO2010105823A1 describes non crystalline form of retigabine as a stable intermediate along with a surface stabilizer and process thereof and a pharmaceutical composition thereof.

International Patent application publication No. WO2011039369A2 describes a stable amorphous solid mixture of retigabine along with at least one pharmaceutically acceptable carrier and a process for the preparation thereof.

Polymorphism is the occurrence of different crystal forms of molecules and molecular complexes. A single molecule, like Retigabine, may give rise to a variety of polymorphs having distinct crystal structures and physical properties like melting point, thermal behavior (e.g., measured by thermogravimetric analysis—“TGA”, or differential scanning calorimetry—“DSC”), X-ray diffraction (XRD) pattern, infrared absorption fingerprint, and/or solid state NMR spectrum. One or more of these techniques may be used to distinguish different polymorphic forms of a compound.

Discovering new polymorphic forms and solvates of a pharmaceutical product can provide materials having desirable processing properties, such as ease of handling, ease of processing, storage stability, and ease of purification or as desirable intermediate crystal finals that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life.

A novel polymorph of a compound may possess physical properties that differ from, and are advantageous over, those of other crystalline or amorphous forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compactibility, handling, flow and blend; and filtration properties. Variations in any one of these properties may affect the chemical and pharmaceutical processing of a compound as well as its bioavailability and may often render the new form advantageous for pharmaceutical and medical use.

The reported crystal modifications A, B, C are less stable and have a tendency to convert under certain conditions to another crystal form with possibly less favorable characteristics.

There remains a need for novel polymorphs of Retigabine with improved properties compared to the crystal modifications reported.

In view of the pharmaceutical value of this compound, a polymorph form of retigabine for use in a medicament should have excellent properties, such as crystallinity, polymorphic stability, chemical stability and process ability to pharmaceutical compositions.

Therefore, there exists a need in the art to provide polymorphs having better solubility, reproducibility and chemical, polymorphic stabilities.

The novel polymorphs of Retigabine disclosed herein have better solubility, reproducibility and chemical, polymorphic stabilities.

### SUMMARY OF THE INVENTION

The present invention relates to novel polymorphs of N-[2-amino-4-(4-fluorobenzylamino)-phenyl]carbamic acid ethyl ester, processes for preparing them, and pharmaceutical composition comprising them.

In one aspect, the present invention provides a novel crystalline polymorph of retigabine designated as crystalline Form I, characterized by XRPD having characteristic peaks at about 4.87, 5.04, 7.03, 9.74, 10.02, 11.6, 18.03, 19.9 and 28.5±0.2 degrees two-theta, which is substantially same as depicted in FIG. 1. The retigabine crystalline Form I is further characterized by an infra red (FTIR) spectrum having main bands at about 705, 712, 736, 747, 775, 789, 826, 839, 859, 885, 936, 946, 980, 1018, 1068, 1087, 1104, 1116, 1161, 1203, 1216, 1246, 1253, 1273, 1306, 1350, 1367, 1393, 1417, 1427, 1440, 1457, 1476, 1508, 1522, 1558, 1568, 1601, 1617, 1701, 1900, 2822, 2851, 2907, 2933, 2945, 2985, 3037, 3140, 3188, 3270, 3376 and 3421±2 cm⁻¹ which is substantially as depicted in FIG. 2.

The retigabine crystalline Form I is further characterized by differential scanning calorimetry (DSC) with an endotherm curve at about 95.06° C. with an onset at about 88.57° C. and with another endotherm curve at about 142.34° C. with an onset at about 140.82° C. measured at 10° C./minutes ramp, substantially in accordance with FIG. 5.

The retigabine crystalline Form I is further characterized by FT Raman spectrum having main bands at about 57, 77, 147, 287, 324, 360, 379, 403, 493, 573, 624, 637, 707, 712, 737, 779, 801, 826, 841, 861, 884, 907, 986, 1010, 1088, 1117, 1160, 1219, 1253, 1272, 1351, 1368, 1426, 1444, 1457, 1478, 1511, 1555, 1603, 1619, 1699, 2869, 2899, 2907, 2934, 2946, 2992, 3008, 3044, 3062±2 cm⁻¹ substantially in accordance with FIG. 7.

In another aspect, the present invention relates to a process for the preparation of retigabine crystalline Form I comprising:
a) providing a solution of retigabine in a solvent or mixture of solvents or aqueous mixtures thereof at elevated temperatures; and
b) evaporating the solvent(s) from the solution of step (a); or
c) cooling the solution of step (a) to below about 20° C.; or
d) adding suitable anti solvent to the solution of step (a) to obtain a saturated solution; and
e) optionally seeding the reaction mixtures of steps (b) to (d) independently; and
f) isolating the solids obtained in steps (b) to (e) independently to afford substantially pure crystalline Form I.

In another aspect, the present invention provides a novel crystalline polymorph of retigabine designated as Form II, characterized by XRPD having characteristic peaks at about 5.07, 10.09, 14.86, 15.1 and 30.4±0.2 degrees two-theta, which is substantially as depicted in FIG. 3. The retigabine crystalline Form II is is further characterized by FTIR spectrum having main bands at about 694, 734, 765, 789, 827, 837, 860, 907, 937, 979, 1014, 1066, 1096, 1155, 1173, 1223, 1259, 1295, 1345, 1367, 1391, 1428, 1474, 1509, 1536, 1600, 1628, 1679, 1709, 1890, 2850, 2873, 2906, 2959, 2985, 3040, 3283, 3346, 3395 and 3442±2 cm⁻¹ which is substantially as depicted in FIG. 4.

The retigabine crystalline Form II is further characterized by differential scanning calorimetric (DSC) thermogram having an endotherm curve at about 141.23° C. with an onset at about 140.48° C., measured at 10° C./minutes ramp, substantially in accordance with FIG. 6.

The retigabine crystalline Form II of the present invention is further characterized by FT Raman spectrum having main bands at about 60, 139, 243, 338, 369, 400, 466, 485, 503, 523, 571, 580, 638, 713, 747, 793, 829, 864, 907, 932, 966, 980, 992, 1015, 1100, 1118, 1157, 1223, 1257, 1296, 1347, 1428, 1450, 1476, 1603, 1619, 1678, 2879, 2942, 2981, 3013, 3022, 3074, 3343±2 cm⁻¹ substantially in accordance FIG. 8.

In yet another aspect, the present invention provides a process for the preparation of retigabine crystalline Form II comprising:
a) providing a solution of retigabine in a solvent or mixture of solvents or aqueous mixtures thereof at elevated temperatures; and
b) evaporating the solvent(s) from the solution of step (a); or
c) cooling the solution of step (a) to above 20° C.; or
d) adding suitable anti-solvent to the solution of step (a) to obtain a saturated solution; and
e) optionally seeding the reaction mixtures of steps (b) to (d) independently; and
f) isolating the solids obtained in steps (b) to (e) independently to afford substantially pure crystalline Form II.

In yet a further aspect, the present invention encompasses a pharmaceutical composition comprising a therapeutically effective amount of retigabine crystalline Form I or Form II and at least a pharmaceutically acceptable carrier.

### BRIEF DESCRIPTION OF THE DRAWINGS

FIG. 1 is a characteristic X-ray powder diffraction pattern of retigabine crystalline Form-I.

FIG. 2 is a characteristic FTIR spectrum of retigabine crystalline Form-I.

FIG. 3 is a characteristic X-ray powder diffraction pattern of retigabine crystalline Form-II.

FIG. 4 is a characteristic FTIR spectrum of retigabine crystalline Form-II.

FIG. 5 is a characteristic Differential Scanning calorimetric (DSC) thermogram of retigabine crystalline Form-I.

FIG. 6 is a characteristic Differential Scanning calorimetric (DSC) thermogram of retigabine crystalline Form-II.

FIG. 7 is a characteristic FT Raman spectrum of retigabine crystalline Form-I.

FIG. 8 is a characteristic FT Raman spectrum of retigabine crystalline Form-II.

### DETAILED DESCRIPTION OF THE INVENTION

The present invention is directed to novel polymorphs of N-[2-amino-4-(4-fluorobenzylamino)-phenyl]carbamic acid ethyl ester, processes for preparing them, and a pharmaceutical composition comprising them.

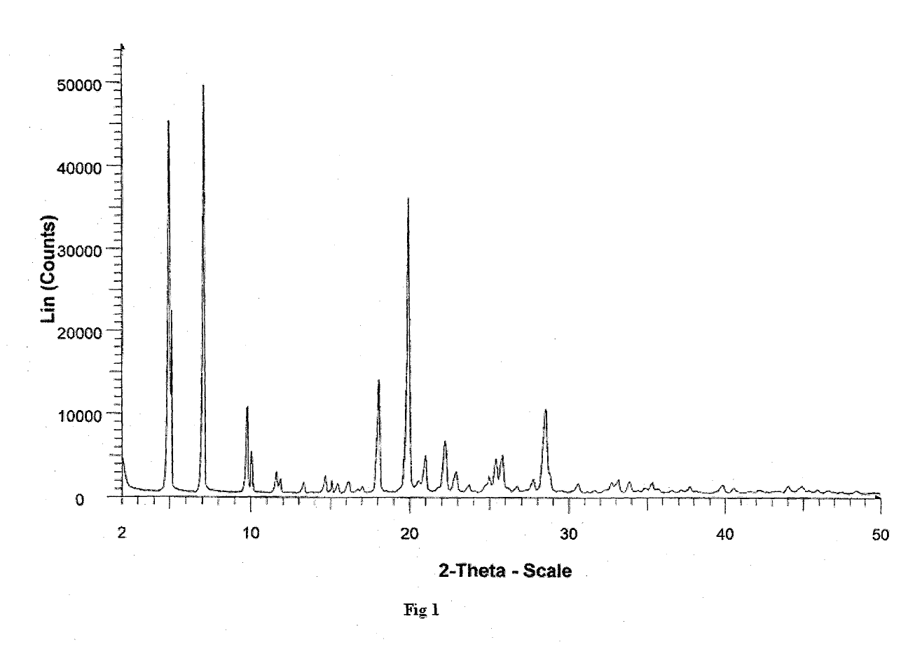

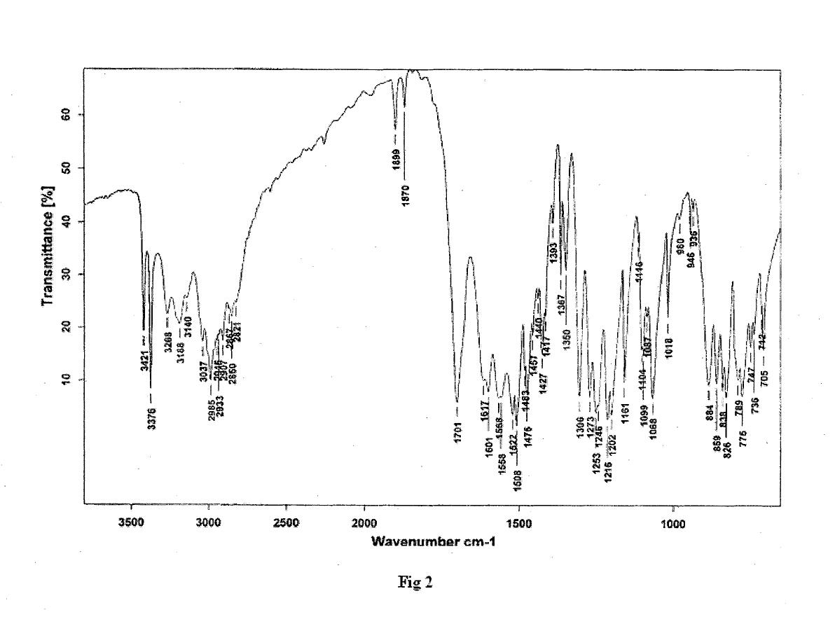

In one embodiment of the present invention, there is provided a novel crystalline polymorph of retigabine designated as crystalline Form I, characterized by XRPD having characteristic peaks at about 4.87, 5.04, 7.03, 9.74, 10.02, 11.6, 18.03, 19.9 and 28.5±0.2 degrees 2-theta, which is substantially as depicted by FIG. 1. The retigabine crystalline Form I is further characterized by FTIR spectrum having main bands at about 705, 712, 736, 747, 775, 789, 826, 839, 859, 885, 936, 946, 980, 1018, 1068, 1087, 1104, 1116, 1161, 1203, 1216, 1246, 1253, 1273, 1306, 1350, 1367, 1393, 1417, 1427, 1440, 1457, 1476, 1508, 1522, 1558, 1568, 1601, 1617, 1701, 1900, 2822, 2851, 2907, 2933, 2945, 2985, 3037, 3140, 3188, 3270, 3376 and 3421±2 cm⁻¹ which is substantially as depicted in FIG. 2.

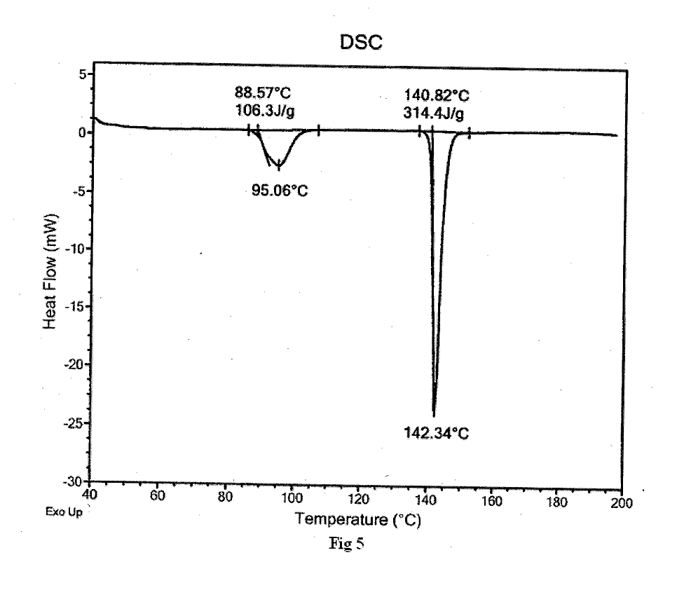

The retigabine crystalline Form I of the present invention is further characterized by a differential scanning calorimetric (DSC) thermogram having an endotherm curve at about 95.06° C. with an onset at about 88.57° C. and with another endotherm curve at about 142.34° C. with an onset at about 140.82° C. measured at 10° C./min. ramp, substantially in accordance with FIG. 5.

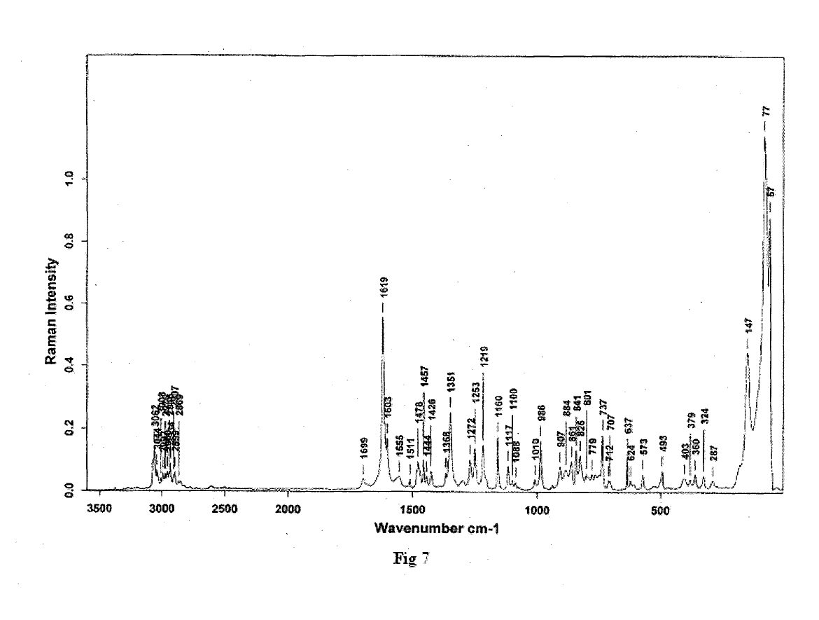

The retigabine crystalline Form I of the present invention is further characterized by FT Raman spectrum comprising peaks at about 57, 77, 147, 287, 324, 360, 379, 403, 493, 573, 624, 637, 707, 712, 737, 779, 801, 826, 841, 861, 884, 907, 986, 1010, 1088, 1117, 1160, 1219, 1253, 1272, 1351, 1368, 1426, 1444, 1457, 1478, 1511, 1555, 1603, 1619, 1699, 2869, 2899, 2907, 2934, 2946, 2992, 3008, 3044, 3062±2 cm⁻¹ which is substantially as depicted in FIG. 7.

In another embodiment of the present invention, there is provided a process for the preparation of retigabine crystalline Form I comprising:
a) providing a solution of retigabine in a solvent or mixture of solvents or aqueous mixtures thereof at elevated temperatures; and
b) evaporating the solvent(s) from the solution of step (a); or
c) cooling the solution of step (a) to below about 20° C.; or
d) adding suitable anti-solvent to the solution of step (a) to obtain a saturated solution; and
e) optionally seeding the reaction mixtures of steps (b) to (d) independently; and
f) isolating the solids obtained in steps (b) to (e) independently to afford substantially pure crystalline Form I.

The solvents that can be used in step (a) include, but are not limited to, water, alcohols such as methanol, ethanol, isopropyl alcohol, n-butanol, isobutyl alcohol, tertiary butyl alcohol and the like; ketonic solvents such as acetone, methyl ethyl ketone, methyl isobutyl ketone, 2-butanone and the like; or a mixture thereof. A most preferable solvent is ethanol.

The temperature required for obtaining a clear and homogenous solution can range from about 45° C. to about 100° C. or the boiling point of the solvent(s) used, preferably from about 50° C. to about boiling point of the solvent(s) used.

The evaporation of solvent(s) in step (b) is carried out at a temperature from about 35° C. to about 75° C. in the presence or absence of vacuum, preferably from 45° C. to about 55° C. in the absence of vacuum.

The cooling of the solution in step (c) can be from about −10° C. to about 50° C., preferably below about 20° C.

The solvents that can be used for the preparation of crystalline Form I by solvent-anti-solvent technique are selected from the group consisting of ketonic solvents such as acetone, methyl ethyl ketone, methyl isobutyl ketone, 2-butanone and the like; esters such as ethyl acetate, isopropyl acetate, butyl acetate and the like or a mixture thereof. A most preferable solvent is ethyl acetate.

The anti-solvents that can be used in step (d) to obtain a saturated solution include, but are not limited to, water, hydrocarbons such as n-hexane, n-heptane, cylcohexane and the like or a mixture thereof. A most preferable anti-solvent is n-hexane.

The temperature suitable for addition of anti-solvent to the solution for obtaining saturated solution is from about 25° C. to about 75° C., preferably from about 30° C. to about 50° C.

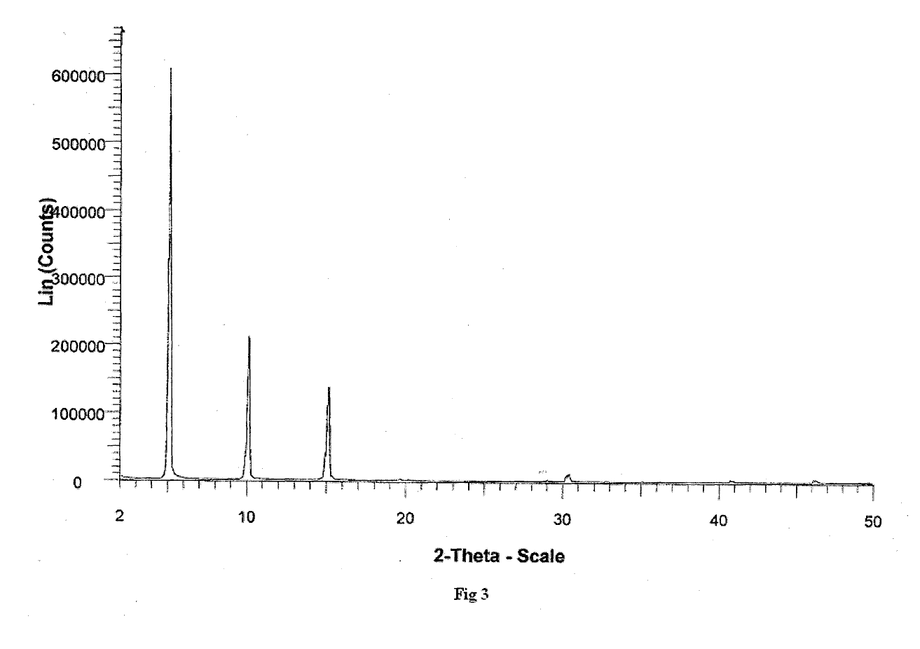

In another embodiment of the present invention, there is provided another novel crystalline polymorph of retigabine designated as Form II, characterized by XRPD having characteristic peaks at about 5.07, 10.09, 14.86, 15.1 and 30.4±0.2 degrees two-theta, which is substantially in accordance with FIG. 3.

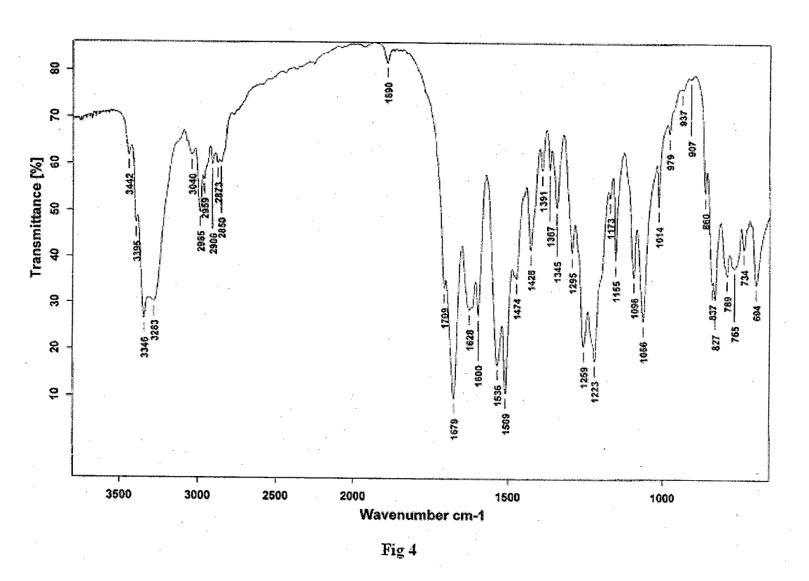

The retigabine crystalline Form II of the present invention is further characterized by FTIR spectrum having main bands at about 694, 734, 765, 789, 827, 837, 860, 907, 937, 979, 1014, 1066, 1096, 1155, 1173, 1223, 1259, 1295, 1345, 1367, 1391, 1428, 1474, 1509, 1536, 1600, 1628, 1679, 1709, 1890, 2850, 2873, 2906, 2959, 2985, 3040, 3283, 3346, 3395 and 3442±2 cm⁻¹ which is substantially as depicted in FIG. 4.

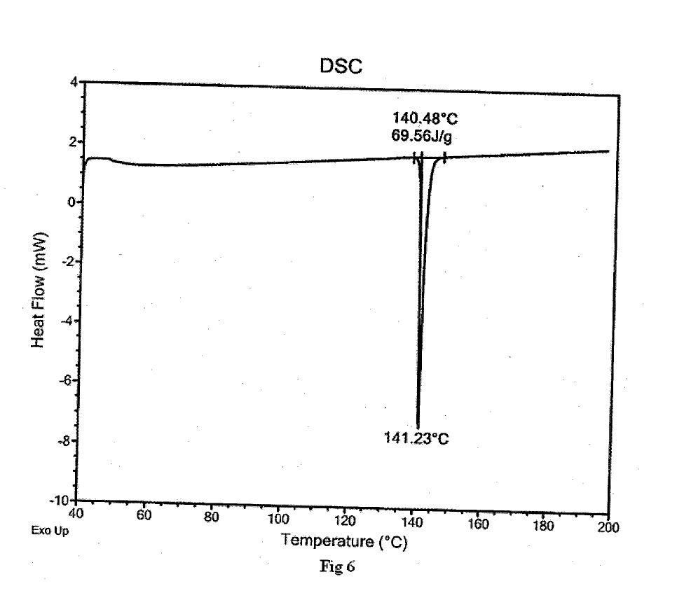

The retigabine crystalline Form II is further characterized by differential scanning calorimetric (DSC) thermogram having an endotherm curve at about 141.23° C. with an onset at about 140.48° C., measured at 10° C./minutes ramp, and is substantially in accordance with FIG. 6.

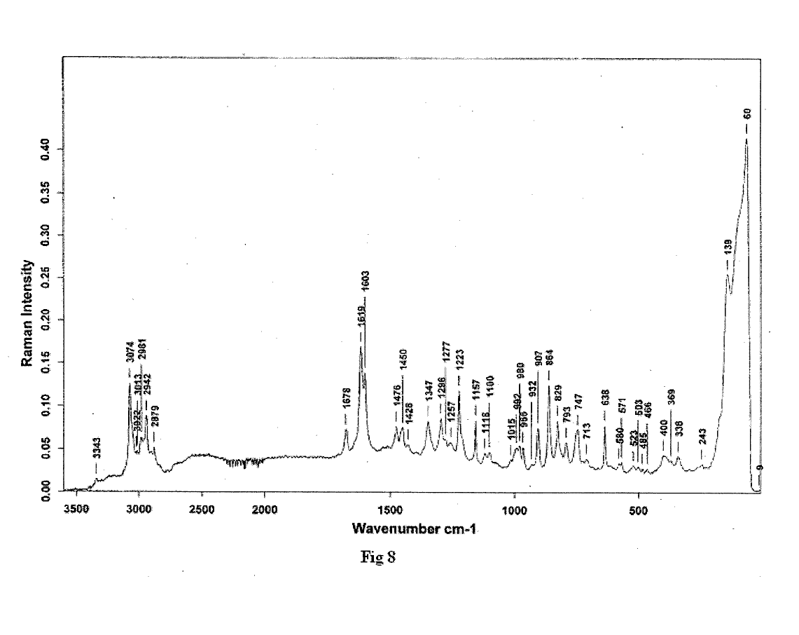

The retigabine crystalline Form II of present invention is further characterized by FT Raman spectrum comprising peaks at about 60, 139, 243, 338, 369, 400, 466, 485, 503, 523, 571, 580, 638, 713, 747, 793, 829, 864, 907, 932, 966, 980, 992, 1015, 1100, 1118, 1157, 1223, 1257, 1296, 1347, 1428, 1450, 1476, 1603, 1619, 1678, 2879, 2942, 2981, 3013, 3022, 3074, 3343±2 cm⁻¹ which is substantially as depicted in FIG. 8.

In yet another embodiment of the present invention, there is provided a process for the preparation of retigabine crystalline Form II, comprising:
a) providing a solution of retigabine in a solvent or mixture of solvents or aqueous mixtures thereof at elevated temperatures; and
b) evaporating the solvent(s) from the solution of step (a); or
c) cooling the solution of step (a) to above 20° C.; or
d) adding suitable anti-solvent to the solution of step (a) to obtain a saturated solution; and
e) optionally seeding the reaction mixtures of steps (b) to (d) independently; and
f) isolating the solids obtained in steps (b) to (e) independently to afford substantially pure crystalline Form II.

The solvents that can be used in step (a) include, but are not limited to, hydrocarbons such as toluene, xylene, n-hexane, n-heptane, cyclohexane and the like; alcohols such as methanol, ethanol, isopropyl alcohol, n-butanol, isobutyl alcohol, tertiary butyl alcohol and the like; or a mixture thereof. A preferable solvent is toluene or ethanol.

The temperature required for obtaining a clear and homogenous solution in step (a) can range from about 45° C. to about 100° C. or the boiling point of the solvent/(s) used, preferably from about 50° C. to about boiling point of the solvent/s used.

The evaporation of solvent(s) in step (b) is carried out at a temperature from about 35° C. to about 75° C. in the presence or absence of vacuum, preferably from 45° C. to about 55° C. in the absence of vacuum.

The cooling of the solution in step (c) is above 20° C., preferably above 35° C.

Optionally seeding of the desired crystalline Form is being used to obtain the desired crystalline form with purity and consistently by adding to the solution of retigabine.

The solvents that can be used for the preparation of crystalline Form II by solvent-anti-solvent technique are selected from the group consisting of aprotic polar solvents such as N,N-dimethyl formamaide (DMF), dimethyl sulfoxide (DMSO), dimethyl acetamide, N-methyl pyrrolidone (NMP), acetonitrile and the like or a mixture thereof; preferably, DMF or DMSO.

The anti-solvents that can be used in step (d) include but are not limited to water, hydrocarbons such as n-hexane, n-heptane, cylcohexane and the like or a mixture thereof. A preferable anti-solvent is water.

The temperature suitable for addition of anti-solvent to the solution for obtaining Form II is typically from about 25° C. to about 75° C., preferably at about 30° C.

As used herein a mixture of solvents refers to a composition comprising more than one solvent.

In one embodiment, the volume of the solvent(s) can be from about 5 to about 150 volumes. Specifically, the volume of the suitable organic solvent may be from about 50 to about 130 volumes, and most specifically the volume of the suitable organic solvent may be from about 80 to about 120 volumes. The mixture may be heated to a temperature sufficient to obtain partial dissolution. The mixture may be heated to a temperature sufficient to obtain complete dissolution.

The solution obtained is optionally filtered through celite or diatomaceous earth to separate the extraneous matter present or formed in the solution by using conventional filtration techniques known in the art.

Evaporation or removal of solvent(s) in the processes described above is accomplished by, for example, substantially complete evaporation of the solvent, concentrating the solution, cooling the solution to obtain crystalline form and filtering the solid under inert atmosphere. Alternatively, the solvent may also be removed by evaporation. Preferably, the method for evaporation or removal of solvent(s) is distillation under reduced pressure.

Crystal growth may be promoted by cooling the solution to a temperature from about −10° C. to about 50° C. Crystal growth may be promoted by cooling the solution to a temperature between about −5° C. to about 40° C. Crystal growth may be promoted by cooling the solution to a temperature between about 0° C. to about 35° C.

Recovery or isolation of retigabine crystalline Form I and Form II obtained by the processes described herein above can be achieved by any conventional methods known in the art, for example, by filtration.

The retigabine substantially in crystalline Form I and Form II obtained by the above processes may be further dried in, for example, vacuum tray dryer, rotocon vacuum dryer, vacuum paddle dryer or pilot plant rotavapor, to further lower residual solvents. When implemented, the preferred instrument is a vacuum tray dryer.

The novel crystalline Form I and Form II of retigabine obtained by the processes of present invention are further dried at a temperature range from about 30° C. to about 75° C. in the presence or absence of vacuum, preferably from about 35° C. to about 55° C.

The novel crystalline forms I and II of retigabine of present invention were characterized by XRPD using a BRUKER AXS D8 FOCUS X-ray Powder diffractometer equipped with Goniometer Type Theta-2Theta; X-Ray source: Copper Kα; Detector: Lynx Eye Detector;
Spinner rotation: 30 deg/min; DIFFRAC^{PLUS} XRD COMMANDER VERSION 2.5.0 with DIFFRAC^{plus} BASIC Evaluation package;

Approximately 1 g of sample was gently flattened on a sample holder and scanned from 2 to 50° two theta, at 0.03° two theta per step and a step time of 0.4 seconds. The sample was simply placed on the sample holder. The sample was rotated at 30 rpm at a voltage 40 KV and current 35 mA.

The novel crystalline forms I and II of retigabine of the present invention were characterized by FTIR using FTIR make: Bruker; FTIR model:VERTEX 70; Resolution: 2 cm⁻¹; Sample scan time: 16 Scans; Background scan Time: 16 Scans; Scan between: 3800 cm−1 and 650 cm⁻¹; OPUS version 6.0;

Sample Preparation:
Weigh about 200.0 mg of KBr, previously dried at 105° C. and cooled, into a mortar and grind to a fine powder, add about 5.0 mg of test sample and mix perfectly and grind to a uniform powder, take a small quantity of the powder and prepare a thin semi-transparent disk, record the IR spectrum of the disk from 3800 cm⁻¹ to 650 cm⁻¹ taking air as reference.

The novel crystalline forms I and II of retigabine of present invention were characterized by FT RAMAN using FT Raman make Bruker, FT Raman model RAM II, Operating Software OPUS 6.0, Resolution 2 cm⁻¹, Sample scan time of 64 Scans, Background scan time 64 Scans, Scan between 3600 cm⁻¹ and 0 cm⁻¹. Raman Laser power for sample 100 mW
(Laser: 9395 cm⁻¹; 500 mW), Detector LN—Ge diode, Beam splitter CaF₂.

Procedure:
Place the test sample onto the v-grooved sample cup and put the sample towards the objective lens assembly in the Raman Compartment.
Record the Raman spectrum of the test sample from 3600 cm⁻¹ to 0 cm⁻¹.

The crystalline Form I and Form II of the present invention are stable with good flow properties, which make them well suitable for formulating retigabine in any dosage form.

The novel crystalline Form I and II of retigabine according to the present invention are substantially stable chemically and polymorphically under stability testing conditions (i.e. 25±2° C. and 75% RH) and in an inert atmosphere.

Advantageously, novel crystalline Forms I and II of retigabine according to the present invention show a better solubility profile compared to the known polymorphs of retigabine (i.e., crystalline modifications A, B, and C).

U.S. Pat. No. 6,538,151 describes three different crystalline forms of retigabine, denominated therein as modifications A, B, and C by XRPD 2-theta, FTIR.

The characteristic XRPD 2-theta peaks of modification A, which are not coinciding with the reflections of the other two modifications (B, C), are 6.97, 18.02 and 19.94° 2-theta.

The characteristic XRPD 2-theta peaks of modification B, which are not coinciding with the reflections of the other two modifications (A, C), are 15.00, 19.29 and 19.58° 2-theta.

The characteristic XRPD 2-theta peaks of modification C, which are not coinciding with the reflections of the other two modifications (A, B), are 9.70 and 21.74° 2-theta.

The characteristic XRPD 2-theta peaks of crystalline Form I which are not coinciding with the reflections of the reported three modifications (A, B, C) are 4.87, 5.04, 7.03, 10.02, 11.6 and 28.5° 2-theta.

The characteristic XRPD 2-theta peaks of crystalline Form II which are not coinciding with the reflections of the reported three modifications (A, B, C) are 5.07, 10.09, 14.86 and 30.4° 2-theta.

Advantageously, the crystalline Forms of the present invention are stable and do not transform into any other crystalline form at any given temperature and pressure.

The compound retigabine used herein as a starting material can be of any polymorph reported or may be crude retigabine or a salt thereof resulting from synthetic processes known in the art, for example, as per the processes described in the U.S. Pat. No. 5,384,330, which is incorporated by reference herein in its entirety.

Advantageously, the novel crystalline Forms of present invention can be interconverted by the processes as exemplified in examples.

The present invention is based in part on the unexpected findings that the new crystalline Forms described herein possesses advantageous physicochemical properties which render its processing as a medicament beneficial. The novel crystalline Forms of the present invention have better solubility thus may have bioavailability as well as desirable stability characteristics enabling their incorporation into a variety of different formulations particularly suitable for pharmaceutical utility. For example, the crystalline forms of the present invention are more soluble than the known crystalline modifications in aqueous vehicles. Thus, the crystalline forms of the present invention may have an improved bioavailability and can be easily formulated to a variety of solid dosage forms.

In yet another embodiment there is provided pharmaceutical compositions comprising at least a therapeutically effective amount of crystalline Form I or Form II of retigabine and at least a pharmaceutically acceptable excepient. Such pharmaceutical compositions may be administered to a mammalian patient for the treatment or prevention of epilepsy in adults in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

The present invention is further illustrated by the following examples, which are not to be construed to be in any way limiting to the present invention, certain modifications and equivalents will be apparent to those skilled in the art and are intended to be included within the scope of the present invention.

### EXAMPLES

### Methods for the Preparation of Retigabine Crystalline Form I

### Example 1

### Preparation of Crystalline Form I by Crystallization

Retigabine (1.5 g) and 6 ml of ethanol were charged into a clean and dry R.B. Flask and heated to about 75° C. The resultant reaction solution was allowed to reach temperature of about 30° C. to crystallize. The resultant reaction suspension was further cooled to about 15° C. The solid separated was filtered and the solid was washed with 3 ml of precooled ethanol. The solid obtained was dried at about 50° C. to afford 1 g of white shiny crystals of Form-I. % Yield: 66% (of theory); Purity by HPLC: 99.6%. Characterized and confirmed by XRPD and IR, DSC which are substantially in accordance with FIGS. 1 and 2, 5.

### Example 2

### Preparation of Crystalline Form-I by Solvent/Antisolvent Technique

Retigabine modification-C (2 g) and 3 ml of ethyl acetate were charged into a clean and dry R.B. Flask and heated to about 60° C. To the resulting reaction solution, 9 ml of n-hexane was added at about 50° C. The resultant reaction suspension was slowly cooled to about 30° C. The solid separated was filtered and the solid was washed with mixture of 1 ml of ethyl acetate and 3 ml of n-hexane. The solid obtained was dried at about 50° C. to afford 1.5 g of crystalline Form-I. % Yield: 75%; Purity by HPLC: 99.6%.

### Example 3

### Preparation of Crystalline Form I Using Acetone and Water

Retigabine modification-C (2 g) and 6 ml of acetone were charged into a clean and dry R.B. Flask, followed by stirring at about 30° C. To the resultant reaction solution 10 ml of water was added drop wise at about 30° C. The resultant reaction suspension was stirred for about 15 minutes. The solid separated was filtered and the solid was washed with a mixture of 1 ml of acetone and 3 ml of water. The solid obtained was dried at about 50° C. to afford 1.3 g of crystalline Form-I. % Yield: 65%.

### Example 4

### Preparation of Crystalline Form I by Evaporation

Retigabine (2 g) and 8 ml of ethanol were charged into a clean and dry R.B. Flask and heated to about 75° C. The resultant reaction solution was allowed to evaporate solvent at about 55° C. The solid separated was isolated and dried at about 50° C. to afford 2 g of white shiny crystals of Form-I.

### Methods for the Preparation of Retigabine Crystalline Form-II

### Example 1

### Preparation of Crystalline Form II

Retigabine (2 g) and 14 ml of toluene were charged into a clean and dry R.B.Flask, followed by heating to reflux. The solution was allowed to reach temperature of about 50° C. to crystallize, then slowly further cooled to about 25° C. The separated solid was filtered and the solid was washed with 4 ml of toluene. The solid obtained was dried at about 50° C. to afford 1.5 g of pure crystalline Form-II (% Yield: 75% of theory; Purity by HPLC: 99.5%). Characterised by XRPD and IR, DSC which are substantially in accordance with FIGS. 3, 4 and 6.

### Example 2

### Preparation of Crystalline Form II Using Crystalline Modification A

Retigabine modification-A (5 g) and 35 ml of toluene were charged into a clean and dry R.B.Flask, followed by heating to about reflux. The resultant reaction solution was allowed to crystallize at about 25° C. The solid separated was filtered and the solid obtained was washed with 10 ml of toluene. The solid obtained was dried at about 50° C. to afford 4.0 g of crystalline Form II. This on further re-crystallized from toluene using same condition as indicated above yielded 4 g of pure form-II (% Yield: 80%).

### Example 3

### Preparation of Crystalline Form II Using Crystalline Modification C

Retigabine modification-C (3 g) and 21 ml of toluene were charged into a clean and dry R.B.Flask, followed by heating to about reflux. The solution was allowed to crystallize at about 60° C. and cooled to about 25° C. The solid separated was filtered and the solid obtained was washed with toluene (6 ml). The solid was dried at about 50° C. to afford 2.5 g of pure form-II. % Yield: 83.3%.

### Example 4

### Preparation of Crystalline Form II Using Ethanol

Retigabine modification-C (2 g) and 8 ml of ethanol were charged into a clean and dry R.B.Flask, followed by heating to about 75° C. The resulting reaction solution was slowly cooled to about 25° C. The separated solid was filtered solid and washed with 2 ml of ethanol. The solid obtained was dried at about 50° C. to afford 2.7 g of crystalline Form-II. % Yield: 90%; Purity by HPLC: 99.5%.

### Example 5

### Preparation of Crystalline Form II Using IPA

Retigabine modification-C (3 g) and 21 ml of Isopropyl alcohol (IPA) were charged into a clean and dry R.B.Flask, followed by heating to about 75° C. The resulting reaction solution was cooled to about 20° C. The separated solid was filtered solid and washed with 6 ml of isopropyl alcohol. The solid obtained was dried at about 50° C. to afford 2.7 g of crystalline Form-II. % Yield: 90%; Purity by HPLC: 99.5%.

### Example 6

### Preparation of Crystalline Form-II Using DMF and Water

Retigabine modification-C (2 g) and 6 ml of N,N-Dimethyl formamide (DMF) were charged into a clean and dry R.B.Flask, followed by stirring at about 30° C. To this 10 ml of water was added drop-wisely at about 30° C. The resultant mixture was stirred for about 15 min. The solid separated was filtered and washed with a mixture of 1 ml DMF and 5 ml water. The solid obtained was dried at about 50° C. to afford 1.5 g of crystalline Form-II. % Yield: 75%.

### Example 7

### Preparation of Crystalline Form-II Using DMSO and Water

Retigabine modification-C (2 g) and 6 ml of Dimethylsulfoxide (DMSO) were charged into a clean and dry R.B.Flask, followed by stirring at about 30° C. To the resultant reaction solution 10 ml of water was added drop-wise at about 30° C. The resultant reaction suspension was stirred at about 30° C. for about 15 min. The solid separated was filtered and washed with a mixture of 1 ml of DMF and 5 ml of water. The solid obtained was dried at about 50° C. to afford 1.7 g of Form-II. % Yield: 80%.

### Example 8

### Preparation of Crystalline Form II by Evaporation

Retigabine (2 g) and 14 ml of toluene were charged into a clean and dry R.B. Flask and heated to about 80° C. The resultant reaction solution was allowed to evaporate solvent at about 55° C. The solid separated was isolated and dried at about 50° C. to afford 2 g of crystals of Form-II.

### Process for Conversion of Crystalline Form II to Form I

Retigabine (2 g) crystalline Form II and 8 ml of ethanol were charged into a clean and dry R.B.Flask, followed by heating to about 75° C. The resultant reaction solution was cooled swiftly to about 0° C. and stirred for about 30 minutes. The solid separated was filtered and the solid was washed with 4 ml of precooled ethanol. The solid obtained was dried at about 50° C. to afford 1.6 g of Form-I. % Yield: 80%; Purity by HPLC: 99.76%.

### Stability of Retigabine Crystalline Form I and II

### Example 1

### Stability of Crystalline Form I

Retigabine crystalline Form I (5 g) was stored at 25° C.±2° C. for 6 weeks in an atmosphere of about 70% RH and the sample was analyzed by XRPD after 6 weeks, crystalline Form I was polymorphically stable at 25° C.±2° C. at a relative humidity (RH) of about 76% for at least 6 weeks.

### Example 2

### Stability of Crystalline Form II

Retigabine crystalline Form II (5 g) was stored at 25° C.±2° C. for 6 weeks in an atmosphere of about 70% RH and the sample was analyzed by XRPD after 6 weeks, crystalline Form II was polymorphically stable at 25° C.±2° C. at a relative humidity (RH) of about 76% for at least 6 weeks.

Comparative solubility profile of crystalline Form I and II along with the
crystalline modifications A, B and C

| | | | | | | | Modification- | | Modification- | | Modification- | |
|---|---|---|---|---|---|---|---|---|---|---|---|---|
| S. No | Diluent | | Form-I | | Form-II | | A | | B | | C | |
| 1 | Water | | <0.1 | mg/mL | <0.1 | mg/mL | <0.1 | mg/mL | <0.1 | mg/mL | <0.1 | mg/mL |
| 2 | pH | 1.2 pH | 10 | mg/mL | 10 | mg/mL | 13 | mg/mL | 10 | mg/mL | 11 | mg/mL |
| | Buffer | 4.5 pH | <0.1 | mg/mL | <0.1 | mg/mL | <0.1 | mg/mL | <0.1 | mg/mL | <0.1 | mg/mL |
| | | 8.5 pH | <0.1 | mg/mL | <0.1 | mg/mL | <0.1 | mg/mL | <0.1 | mg/mL | <0.1 | mg/mL |
| 3 | Solvent | Alcohol | 25 | mg/mL | 24 | mg/mL | 23 | mg/mL | 22 | mg/mL | 16 | mg/mL |
| | | (96%) | | | | | | | | | | |
| | | Acetone | 290 | mg/mL | 250 | mg/mL | 280 | mg/mL | 280 | mg/mL | 310 | mg/mL |
| | | Methylene | 40 | mg/mL | 70 | mg/mL | 50 | mg/mL | 80 | mg/mL | 50 | mg/mL |
| | | chloride | | | | | | | | | | |
| | | DMSO | 700 | mg/mL | 700 | mg/mL | 800 | mg/mL | 700 | mg/mL | 700 | mg/mL |

1-14. (canceled)

15. Crystalline Form II of retigabine characterized by an X-ray powder diffraction (XRPD) pattern having characteristic 2-theta peaks at about 5.07, 10.09, 14.86, 15.1 and 30.4±0.2 degrees substantially in accordance with FIG. 3.

16. (canceled)

17. The crystalline Form II of claim 15, which is further characterized by an FT-IR spectrum having main bands at about 694, 734, 765, 789, 827, 837, 860, 907, 937, 979, 1014, 1066, 1096, 1155, 1173, 1223, 1259, 1295, 1345, 1367, 1391, 1428, 1474, 1509, 1536, 1600, 1628, 1679, 1709, 1890, 2850, 2873, 2906, 2959, 2985, 3040, 3283, 3346, 3395 and 3442±2 cm⁻¹ substantially in accordance with FIG. 4.

18. The crystalline Form II of claim 15, which is further characterized by differential scanning calorimetric (DSC) thermogram having an endotherm curve at about 141.23° C. with an onset at about 140.48° C. substantially in accordance with FIG. 6.

19. The crystalline Form II of claim 15, which is further characterized by FT-RAMAN spectra having peaks at about 60, 139, 243, 338, 369, 400, 466, 485, 503, 523, 571, 580, 638, 713, 747, 793, 829, 864, 907, 932, 966, 980, 992, 1015, 1100, 1118, 1157, 1223, 1257, 1296, 1347, 1428, 1450, 1476, 1603, 1619, 1678, 2879, 2942, 2981, 3013, 3022, 3074, 3343±2 cm⁻¹ which is substantially as depicted in FIG. 8.

20-29. (canceled)

30. A process for the preparation of retigabine crystalline Form II of claim 15, comprising:
a) providing a solution of retigabine in a solvent selected from the group consisting of toluene, ethanol, isopropyl alcohol and mixtures thereof at a temperature of about 50° C. to about the boiling temperature of the solvent used;
b) cooling the solution obtained in step-(a) to a temperature of about 20° C. to about 35° C. to cause crystallization; and
c) isolating the substantially pure crystalline Form II of retigabine obtained in step-(b).

31. A process for the preparation of retigabine crystalline Form II of claim 15, comprising:
a) providing a solution of retigabine in N,N-dimethylformamide or dimethylsulfoxide;
b) adding water (anti-solvent) to the solution obtained in step-(a) to cause crystallization; and
c) isolating the substantially pure crystalline Form II of retigabine obtained in step-(b).
